(19) United States
(12) Patent Application Publication
(10) Pub. No.: US 2025/0002559 A1
(43) Pub. Date: Jan. 2, 2025

(54) PHARMACEUTICAL FORMULATION FOR TREATMENT OF CEREBRAL STROKE

(71) Applicant: PHARMAZZ, INC., Willowbrook, IL (US)

(72) Inventor: Anil Gulati, Naperville, IL (US)

(73) Assignee: PHARMAZZ, INC., Willowbrook, IL (US)

(21) Appl. No.: 18/343,087

(22) Filed: Jun. 28, 2023

(51) Int. Cl. C07K 14/72; A61K 31/047; A61K 31/7068; A61P 25/28

(52) U.S. Cl. CPC C07K 14/72; A61K 31/047; A61K 31/7068; A61P 25/28

(57) ABSTRACT

A pharmaceutical formulation for treatment of cerebral stroke includes an endothelin-B receptor analog in a dosing range of 0.00001 mg/kg−1 mg/kg, and a drug in a dosing range of 0.0015 g/kg-5 g/kg, wherein endothelin-B receptor analog includes, but not limited to sovateltide, BQ-3020, [Ala^{1,3,11,15}]-endothelin, sarafotoxin S6c, endothelin-3, and the drug includes, but not limited to mannitol and citicoline.

### FIELD

The aspects of the disclosed embodiments relate to a pharmaceutical formulation for treatment of cerebral stroke that is capable of being administered through intravenous or intra-arterial route for treatment of various neurological conditions such as cerebral ischemic stroke, intracranial haemorrhage.

### BACKGROUND

Neurological conditions such as cerebral ischemic stroke, intracranial haemorrhage, and brain injury commonly cause cerebral edema. Stroke is one of the most common causes of death and is also an important cause of serious, long-lasting disability. Ischemic stroke caused by arterial occlusion is responsible for most strokes. Various treatment options have been developed for treatment of strokes. Mannitol is one such treatment option as it decreases brain volume by reducing overall water content, blood volume by vasoconstriction, and cerebral spinal fluid (CSF) volume by decreasing water content. Mannitol may also improve cerebral perfusion by reducing viscosity or altering red blood cell rheology.

However, administration of mannitol alone is insufficient to reduce the morbidity and mortality of patients with cerebral stroke. Moreover, monitoring of fluids and electrolytes, serum osmolarity, and renal, cardiac, and pulmonary function needs to be carried out during and following mannitol infusion, which makes the treatment a cumbersome process.

Another treatment option available for treatment of stroke is usage of tissue plasminogen activator (tPA). However, the use of tPA is limited to a short time window of <4.5 h from the onset of symptoms, and it also has a risk of intracranial haemorrhage.

U.S. Pat. No. 8,623,823 discloses methods of using an ET_{B} receptor agonist, such as IRL-1620, for the treatment of stroke or cerebrovascular accidents are disclosed. The ET_{B} receptor agonist is used alone or in combination with a second agent useful in the treatment of stroke or other cerebrovascular accident.

U.S. Ser. No. 10/561,704 discloses compositions and methods for treating neuropsychiatric disorders in vertebrates and humans. More specifically, the present invention provides for use of IRL-1620, an endothelin-B receptor agonist, in appropriate doses to be a neuroprotective and a neuroregenerative agent. Accordingly, in one aspect the disclosure provides a method of treating a neuropsychiatric disorder comprising administering to a patient in need thereof a therapeutically effective amount of an endothelin-B receptor agonist to treat the neuropsychiatric disorder. In some embodiments, the endothelin-B receptor agonist is co-administered with an additional agent to treat the neuropsychiatric disorder. In some embodiments, the additional agent is selected from the group consisting of an antidepressant, an anti-inflammatory agent, a CNS stimulant, a neuroleptic, and an anti-proliferative agent.

Conventionally, many compositions and methods have been developed that are capable of treating various neurological conditions. However, since such compositions and methods employs only a single drug, the neurological outcomes of such compositions are not sufficient.

In order to overcome the aforementioned drawbacks, there exists a need in the art to develop a formulation that includes at least two pharmaceutically active ingredients which increases overall efficacy of the formulation for treating various neurological conditions such as cerebral ischemic stroke, intracranial haemorrhage.

The aspects of the disclosed embodiments are directed to overcoming the disadvantages of the prior art.

to the aspects of the disclosed embodiments provide a formulation that is capable of treating various neurological conditions such as cerebral ischemic stroke, intracranial haemorrhage.

Another aspect of the disclosed embodiments provides a formulation that includes at least two pharmaceutically active ingredients for obtaining an improved pharmaceutical action.

A further aspect of the disclosed embodiments provides a formulation that is capable of being administered through intravenous or intra-arterial route.

The foregoing and other aspects, features, and advantages disclosed embodiments will become readily apparent upon further review of the following detailed description of the preferred embodiment as illustrated in the accompanying drawings.

### SUMMARY

The aspects of the disclosed embodiments relate to a pharmaceutical formulation for treatment of cerebral stroke that is capable of being administered through intravenous or intra-arterial route and works on a synergistic effect of at least two pharmaceutically active ingredients in view of treating various neurological conditions.

According to an embodiment, a pharmaceutical formulation for treatment of cerebral stroke comprises of: i) an endothelin-B receptor analog in a dosing range of 0.00001 mg/kg−1 mg/kg, and ii) a drug in a dosing range of 0.0015 g/kg-5 g/kg.

According to another embodiment, the endothelin-B receptor analog includes, but not limited to sovateltide, BQ-3020, [Ala^{1,3,11,15}]-endothelin, sarafotoxin S6c, endothelin-3, and the drug includes, but not limited to mannitol and citicoline.

While the aspects of the disclosed embodiments has been described and shown with particular reference to the preferred embodiment, it will be apparent that variations might be possible that would fall within the scope of the aspects of the disclosed embodiments.

### BRIEF DESCRIPTION OF THE DRAWINGS

These and other features, aspects, and advantages of the disclosed embodiments will become better understood with regard to the following description, appended claims, and accompanying drawings where:

FIG. 1 illustrates a graphical representation of modified Rankin scale of patients treated with control and sovateltide along with mannitol;

FIG. 2 illustrates a graphical representation of number of cerebral ischemic stroke patients treated with control and sovateltide along with mannitol and having a modified Rankin Scale of 0-2 at 90 days post-randomization;

FIG. 3 illustrates a graphical representation of number of cerebral ischemic stroke patients treated with control and sovateltide along with mannitol and having improvement of 2 or more modified Rankin Scale from baseline at 90 days post-randomization;

FIG. 4 illustrates a graphical representation of modified Rankin scale of patients treated with control and sovateltide along with citicoline;

FIG. 5 illustrates a graphical representation of number of cerebral ischemic stroke patients treated with control and sovateltide along with citicoline and having a modified Rankin Scale of 0-2 at 90 days post-randomization;

FIG. 6 illustrates a graphical representation of number of cerebral ischemic stroke patients treated with control and sovateltide along with citicoline and having improvement of 2 or more modified Rankin Scale from baseline at 90 days post-randomization;

FIG. 7 illustrates a graphical of modified Rankin scale of patients treated with control and sovateltide along with cerebrolysin;

FIG. 8 illustrates a graphical representation of number of cerebral ischemic stroke patients treated with control and sovateltide along with cerebrolysin and having a modified Rankin Scale of 0-2 at 90 days post-randomization;

FIG. 9 illustrates a graphical representation of number of cerebral ischemic stroke patients treated with control and sovateltide along with cerebrolysin and having improvement of 2 or more modified Rankin Scale from baseline at 90 days post-randomization;

FIG. 10 illustrates a graphical representation of modified Rankin scale of patients treated with control and sovateltide along with thrombolytics;

FIG. 11 illustrates a graphical representation of number of cerebral ischemic stroke patients treated with control and sovateltide along with thrombolytics and having a modified Rankin Scale of 0-2 at 90 days post-randomization; and

FIG. 12 illustrates a graphical representation of number of cerebral ischemic stroke patients treated with control and sovateltide along with thrombolytics and having improvement of 2 or more modified Rankin Scale from baseline at 90 days post-randomization.

### DETAILED DESCRIPTION

The following description includes the preferred best mode of one embodiment aspects of the disclosed embodiments. It will be clear from this description that the aspects of the disclosed embodiments are not limited to these illustrated embodiments but can also includes a variety of modifications and embodiments thereto. Therefore, the present description should be seen as illustrative and not limiting. While the aspects of the disclosed embodiments are susceptible to various modifications and alternative constructions, it should be understood, that there is no intention to limit the aspects of the disclosed embodiments to the specific form disclosed, but, on the contrary, the aspects of the disclosed embodiments can cover all modifications, alternative constructions, and equivalents falling within the spirit and scope of the disclosed embodiments as defined in the claims.

In any embodiment described herein, the open-ended terms “comprising,” “comprises,” and the like (which are synonymous with “including,” “having” and “characterized by”) may be replaced by the respective partially closed phrases “consisting essentially of,” consists essentially of,” and the like or the respective closed phrases “consisting of,” “consists of, the like.

As used herein, the singular forms “a,” “an,” and “the” designate both the singular and the plural, unless expressly stated to designate the singular only.

The aspects of the disclosed embodiments relate to a pharmaceutical formulation for treatment of cerebral stroke that includes an endothelin-B receptor analog and a drug, wherein the analog shows synergistic effect with the drug, resulting in enhanced therapeutic action of the formulation against various neurological conditions such as stroke.

According to an embodiment, a pharmaceutical formulation for treatment of cerebral stroke, comprising: an endothelin-B receptor analog in a dosing range of 0.00001 mg/kg−1 mg/kg, and a drug in a dosing range of 0.0015 g/kg-5 g/kg.

The endothelin-B receptor analog discloses herein is selected from a group comprising sovateltide, BQ-3020, [Ala^{1,3,11,15}]-endothelin, sarafotoxin S6c, endothelin-3. The drug disclosed herein includes, but not limited to mannitol and citicoline.

The proposed formulation is a combination of mannitol and sovateltide or citicoline and sovateltide. Herein, the mannitol dose range is 0.1 to about 5.0 g/kg, sovateltide dose range is 0.00001 mg/kg to about 1 mg/kg, and the citicoline dose range is 1.5 mg/kg to about 75 mg/kg. The formulation herein is prepared as a lyophilized formulation and is capable of being administered through intravenous or intra-arterial route.

In an embodiment, the formulation is administered through a catheter with or without adjunctive mechanical methods or during the procedure of embolectomy or thrombectomy.

In another embodiment, the formulation is prepared in the form of nanoparticle, gel or hydrogel, nano-emulsion, microparticle, colloidal suspension, sterile suspension, solution, aerosol, and powder.

In another embodiment, the formulation comprises a biodegradable polymer, wherein the biodegradable polymer is poly(lactic-co-glycolic acid) (PLGA) or pegylated PLGA (PEG-PLGA).

In another embodiment, the formulation includes additives, including but not limited to polyvinyl alcohol (PVA) or other known nanoparticle stabilizers.

### EXAMPLE

The pharmaceutical formulation for treatment of cerebral stroke comprises of 0.0003 mg/kg of sovateltide and 0.75 to 1.25 g/kg of mannitol or 0.0003 mg/kg of sovateltide and 15 to 30 mg/kg of citicoline.

For administration of the proposed formulation comprising mannitol and sovateltide, mannitol infusion is followed by administration of sovateltide. Similarly, for administration of the proposed formulation comprising citicoline and sovateltide, citicoline infusion is followed by administration of sovateltide.

According to another embodiment, sovateltide is administered before, after, or simultaneously with mannitol or citicoline.

Accordingly, to another embodiment, an example of treatment with mannitol and sovateltide is as follows: the treatment is initiated upon admission of a patient with a neurological deficit, and mannitol solution is administered intravenously. The dose may range from 0.1 g/kg to 5.0 g/kg daily. The average dose of mannitol during the 24 hours of administration is about 0.75 to 1.25 g/kg, and the duration of mannitol treatment can continue for about 3 to 10 days, with most patients receiving mannitol for about six days. Another way to administer mannitol is to infuse 100 ml of 20% mannitol given every four hours for five to six days. Sovateltide is administered in the dose of 0.3 μg/kg as an intravenous bolus over one minute at multiple intervals.

Accordingly, to another embodiment, an example of treatment with citicoline and sovateltide is as follows: the treatment is initiated within twenty-four hours after the onset of symptoms of neurological deficits. The citicoline administration is done in a dose ranging from 1.5 mg/kg to 75 mg/kg intravenously every twelve hours during the first three to six days and then orally for six weeks in a dose of 1000 grams every twelve hours. Sovateltide is to be administered in the dose of 0.3 μg/kg as an intravenous bolus over one minute at multiple intervals.

A prospective, multicentric, randomized phase III study was conducted in acute cerebral ischemic stroke patients aged 18 years through 78 years. Patients with radiologic confirmation of ischemic stroke could be enrolled if presenting up to 24 hours after the onset of symptoms and with a modified Rankin Score (mRS) of 3 to 4 and an NIHSS (National Institute of Health Stroke Scale) score of greater than 5. Patients with intracranial haemorrhage and those receiving endovascular therapy were excluded.

Patients were randomized in a 1:1 ratio, 80 patients to the sovateltide group and 78 patients to the control (saline) group. Every patient received the standard of care (SOC) and was followed for 90 days. The study drug (normal saline or sovateltide (0.3 μg/kg) was administered in three doses administered as an intravenous bolus over 1 minute at an interval of 3 hours±1 hour on day 1, day 3, and day 6 (total sovateltide dose of 0.9 μg/kg/day).

The primary objectives were to determine the neurological outcome based on modified Rankin Scale (mRS) score, National Institute of Health Stroke Scale (NIHSS) score, and Barthel Index (BI) scale score from day 1 through day 90. In addition, quality-of-Life was measured by EuroQol-EQ-5D (EQ-5D) and stroke-specific quality-of-life (SSQoL) scores at 60 days and 90 days of treatment.

A total of 158 patients with acute cerebral ischemic stroke were enrolled in this test, of which 137 completed 90-day follow-up. Patients received saline (n=70; 45 male and 25 female) or sovateltide (n=67; 45 male and 22 female) within 24 hours of the onset of stroke. Patients received the investigational drug (mannitol or citicoline) at 16.85±0.74 and 17.40±0.67 hours (mean±SEM, p=0.583) of stroke onset in control and sovateltide cohorts, respectively. The baseline characteristics and SOC in both cohorts were similar. Alberta Stroke Program Early Computed Tomographic Score (ASPECTS) mean value was similar in the control (7.44) and sovateltide (7.61) groups indicating that the extent of infarction was similar in both the groups.

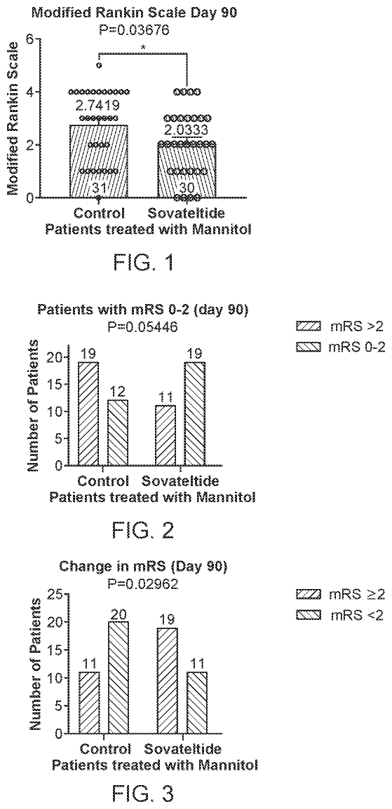

The modified Rankin Scale at post-randomization day 90 of control and sovateltide cerebral ischemic stroke patients (N=61) treated with mannitol was determined in 31 patients of the control group and 30 patients in the sovateltide group. Referring to FIG. 1 and table 1, a graphical and a tabular representation of modified Rankin scale of patients treated with control and sovateltide along with mannitol are illustrated, respectively. It was found that patients treated with mannitol and sovateltide had significantly lower modified Rankin Scale compared to those that received mannitol.

TABLE 1
Modified Rankin scale of patients treated
with control and sovateltide along with mannitol
Modified Rankin Scale Control vs Sovateltide Day 90 Mannitol

| | |
|---|---|
| Column B | Sovateltide |
| vs. | vs. |
| Column A | Control |

Unpaired t test

| | |
|---|---|
| P value | 0.0368 |
| P value summary | * |
| Significantly different (P < 0.05)? | Yes |
| One- or two-tailed P value? | Two-tailed |
| t, df | t = 2.137, df = 59 |
| Mean of column A | 2.742 |
| Mean of column B | 2.033 |
| Difference between means (B − A) ± SEM | −0.7086 ± 0.3316 |
| 95% confidence interval | −1.372 to −0.04509 |
| R squared (eta squared) | 0.07184 |

Data analyzed

| | |
|---|---|
| Sample size, column A | 31 |
| Sample size, column B | 30 |

Referring to FIG. 2 and table 2, a graphical representation and a tabular representation of number of cerebral ischemic stroke patients treated with control and sovateltide along with mannitol and having a modified Rankin Scale of 0-2 at 90 days post-randomization are illustrated, respectively. The number of cerebral ischemic stroke patients with a modified Rankin Scale of 0-2 at 90 days post-randomization was significantly more in those treated with mannitol and an endothelin-B agonist, sovateltide, compared to those with mannitol treatment.

TABLE 2
Number of cerebral ischemic stroke patients
treated with control and sovateltide along
with mannitol and having a modified Rankin Scale
of 0-2 at 90 days post-randomization
Number of Patients with mRS of 0-2 day 90 Mannitol
P value and statistical significance

| Test | Chi-square | |
|---|---|---|
| Chi-square, df | 3.699, 1 | |
| Z | 1.923 | |
| P value | 0.0545 | |
| P value summary | ns | |
| One- or two-sided | Two-sided | |
| Statistically significant | No | |
| (P < 0.05)? | | |
| Effect size | Value | 95% CI |
| Relative Risk | 1.672 | 0.9905 to 2.963 |
| Reciprocal of relative risk | 0.5982 | 0.3375 to 1.010 |
| Odds ratio | 2.735 | 0.9381 to 7.734 |
| Reciprocal of odds ratio | 0.3657 | 0.1293 to 1.066 |

Methods used to compute CIs

| | | |
|---|---|---|
| Relative Risk | Koopman asymptotic score | |
| Odds ratio | Baptista-Pike | |
| Data analyzed | mRS > 2 | mRS 0-2 |
| Control | 19 | 12 |
| Sovateltide | 11 | 19 |
| Total | 30 | 31 |
| Percentage of row total | mRS > 2 | mRS 0-2 |
| Control | 61.29% | 38.71% |
| Sovateltide | 36.67% | 63.33% |

Referring to FIG. 3 and table 3, a graphical representation and a tabular representation of number of cerebral ischemic stroke patients treated with control and sovateltide along with mannitol and having improvement of 2 or more modified Rankin Scale from baseline at 90 days post-randomization are illustrated, respectively. The number of cerebral ischemic stroke patients with an improvement of 2 or more modified Rankin Scale from baseline at 90 days post-randomization was significantly more in the group treated with mannitol and an endothelin-B agonist, sovateltide compared to the group treated with mannitol. Therefore, sovateltide improved the neurological outcome of mannitol.

TABLE 3
Number of cerebral ischemic stroke patients treated
with control and sovateltide along with mannitol
and having improvement of 2 or more modified Rankin
Scale from baseline at 90 days post-randomization
Change in mRS of 2 or more from day 1 to day 90 Mannitol
P value and statistical significance

| Test | Chi-square | |
|---|---|---|
| Chi-square, df | 4.731, 1 | |
| Z | 2.175 | |
| P value | 0.0296 | |
| P value summary | * | |
| One- or two-sided | Two-sided | |
| Statistically significant | Yes | |
| (P < 0.05)? | | |
| Effect size | Value | 95% CI |
| Relative Risk | 0.5603 | 0.3163 to 0.9456 |
| Reciprocal of | 1.785 | 1.058 to 3.161 |
| relative risk | | |
| Odds ratio | 0.3184 | 0.1093 to 0.9381 |
| Reciprocal of odds ratio | 3.140 | 1.066 to 9.149 |

Methods used to compute CIs

| | | |
|---|---|---|
| Relative Risk | Koopman asymptotic score | |
| Odds ratio | Baptista-Pike | |
| Data analyzed | mRS ≥ 2 | mRS < 2 |
| Control | 11 | 20 |
| Sovateltide | 19 | 11 |
| Total | 30 | 31 |
| Percentage of row total | mRS ≥ 2 | mRS < 2 |
| Control | 35.48% | 64.52% |
| Sovateltide | 63.33% | 36.67% |

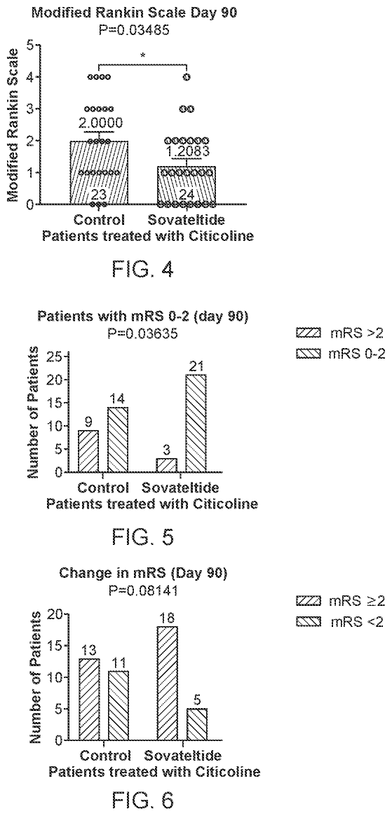

The modified Rankin Scale at post-randomization day 90 of control and sovateltide cerebral ischemic stroke patients (N=47) treated with citicoline was determined in 23 patients of the control group and 24 patients in the sovateltide group. Referring to FIG. 4 and table 4, a graphical and a tabular representation of modified Rankin scale of patients treated with control and sovateltide along with citicoline are illustrated, respectively. It was found that patients treated with citicoline and an endothelin-B agonist, sovateltide, had significantly better neurological outcomes with significantly lower modified Rankin Scale compared to those that received citicoline.

TABLE 4
Modified Rankin scale of patients treated
with control and sovateltide along with citicoline
Modified Rankin Scale Control vs Sovateltide Day 90 Citicoline

| | |
|---|---|
| Column B | Sovateltide |
| vs. | vs. |
| Column A | Control |

Unpaired t test

| | |
|---|---|
| P value | 0.0348 |
| P value summary | * |
| Significantly different (P < 0.05)? | Yes |
| One- or two-tailed P value? | Two-tailed |
| t, df | t = 2.176, df = 45 |
| Mean of column A | 2.000 |
| Mean of column B | 1.208 |
| Difference between | −0.7917 ± 0.3638 |
| means (B − A) ± | |
| SEM | |
| 95% confidence interval | −1.524 to −0.05888 |
| R squared (eta squared) | 0.09520 |

Data analyzed

| | |
|---|---|
| Sample size, column A | 23 |
| Sample size, column B | 24 |

Referring to FIG. 5 and table 5, a graphical representation and a tabular representation of number of cerebral ischemic stroke patients treated with control and sovateltide along with citicoline and having a modified Rankin Scale of 0-2 at 90 days post-randomization are illustrated, respectively. The number of cerebral ischemic stroke patients with a modified Rankin Scale of 0-2 at 90 days post-randomization was significantly more in those treated with citicoline and an endothelin-B agonist, sovateltide compared to those with citicoline treatment.

TABLE 5
Number of cerebral ischemic stroke patients
treated with control and sovateltide along with
citicoline and having a modified Rankin Scale
of 0-2 at 90 days post-randomization
Number of Patients with mRS of 0-2 day 90 Citicoline
P value and statistical significance

| Test | Chi-square | |
|---|---|---|
| Chi-square, df | 4.381, 1 | |
| z | 2.093 | |
| P value | 0.0363 | |
| P value summary | * | |
| One- or two-sided | Two-sided | |
| Statistically significant | Yes | |
| (P < 0.05)? | | |
| Effect size | Value | 95% CI |
| Relative Risk | 3.130 | 1.067 to 9.842 |
| Reciprocal of | 0.3194 | 0.1016 to 0.9376 |
| relative risk | | |
| Odds ratio | 4.500 | 1.058 to 17.02 |
| Reciprocal of odds ratio | 0.2222 | 0.05875 to 0.9455 |

Methods used to compute CIs

| | | |
|---|---|---|
| Relative Risk | Koopman asymptotic score | |
| Odds ratio | Baptista-Pike | |
| Data analyzed | mRS > 2 | mRS 0-2 |
| Control | 9 | 14 |
| Sovateltide | 3 | 21 |
| Total | 12 | 35 |
| Percentage of row total | mRS > 2 | mRS 0-2 |
| Control | 39.13% | 60.87% |
| Sovateltide | 12.50% | 87.50% |

Referring to FIG. 6 and table 6, a graphical representation and a tabular representation of number of cerebral ischemic stroke patients treated with control and sovateltide along with citicoline and having improvement of 2 or more modified Rankin Scale from baseline at 90 days post-randomization are illustrated, respectively. The number of cerebral ischemic stroke patients with an improvement of 2 or more modified Rankin Scale from baseline at 90 days post-randomization was significantly more in the group treated with citicoline and an endothelin-B agonist, sovateltide compared to the group treated with citicoline. Therefore, sovateltide improved the neurological outcome of citicoline.

TABLE 6
Number of cerebral ischemic stroke patients treated
with control and sovateltide along with citicoline
and having improvement of 2 or more modified Rankin Scale
from baseline at 90 days post-randomization
Change in mRS of 2 or more from day 1 to day 90 Citicoline
P value and statistical significance

| Test | Chi-square | |
|---|---|---|
| Chi-square, df | 3.037, 1 | |
| z | 1.743 | |
| P value | 0.0814 | |
| P value summary | ns | |
| One- or two-sided | Two-sided | |
| Statistically significant | No | |
| (P < 0.05)? | | |
| Effect size | Value | 95% CI |
| Relative Risk | 0.6921 | 0.4326 to 1.049 |
| Reciprocal of | 1.445 | 0.9535 to 2.311 |
| relative risk | | |
| Odds ratio | 0.3283 | 0.09738 to 1.121 |
| Reciprocal of odds ratio | 3.046 | 0.8924 to 10.27 |

Methods used to compute CIs

| | | |
|---|---|---|
| Relative Risk | Koopman asymptotic score | |
| Odds ratio | Baptista-Pike | |
| Data analyzed | mRS ≥ 2 | mRS < 2 |
| Control | 13 | 11 |
| Sovateltide | 18 | 5 |
| Total | 31 | 16 |
| Percentage of row total | mRS ≥ 2 | mRS < 2 |
| Control | 54.17% | 45.83% |
| Sovateltide | 78.26% | 21.74% |

Accordingly to another embodiment, a formulation comprising sovateltide and cerebrolysin was also prepared. The modified Rankin Scale at post-randomization day 90 of control and sovateltide cerebral ischemic stroke patients (N=26) treated with cerebrolysin was determined in 14 patients of the control group and 12 patients in the sovateltide group.

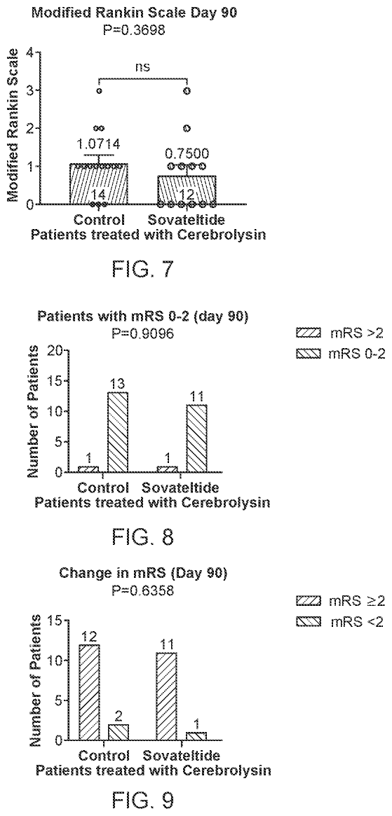

Referring to FIG. 7 and table 7, a graphical and a tabular representation of modified Rankin scale of patients treated with control and sovateltide along with cerebrolysin are illustrated, respectively. It was found that patients treated with cerebrolysin and an endothelin-B agonist, sovateltide, had similar neurological outcomes compared to those that received cerebrolysin.

TABLE 7
Modified Rankin scale of patients treated with
control and sovateltide along with cerebrolysin
Modified Rankin Scale Control vs Sovateltide Day 90 Cerebrolysin

| | |
|---|---|
| Column B | Sovateltide |
| vs. | vs. |
| Column A | Control |

Unpaired t test

| | |
|---|---|
| P value | 0.3698 |
| P value summary | ns |
| Significantly different (P < 0.05)? | No |
| One- or two-tailed P value? | Two-tailed |
| t, df | t = 0.9140, df = 24 |
| Mean of column A | 1.071 |
| Mean of column B | 0.7500 |
| Difference between means | −0.3214 ± 0.3517 |
| (B − A) ± SEM | |
| 95% confidence interval | −1.047 to 0.4044 |
| R squared (eta squared) | 0.03364 |

Data analyzed

| | |
|---|---|
| Sample size, column A | 14 |
| Sample size, column B | 12 |

Referring to FIG. 8 and table 8, a graphical representation and a tabular representation of number of cerebral ischemic stroke patients treated with control and sovateltide along with cerebrolysin and having a modified Rankin Scale of 0-2 at 90 days post-randomization are illustrated, respectively. The number of cerebral ischemic stroke patients with a modified Rankin Scale of 0-2 at 90 days post-randomization was also similar in those treated with cerebrolysin and an endothelin-B agonist, sovateltide compared to those with cerebrolysin treatment.

TABLE 8
Number of cerebral ischemic stroke patients
treated with control and sovateltide along with
cerebrolysin and having a modified Rankin Scale
of 0-2 at 90 days post-randomization
Number of Patients with mRS of 0-2 day 90 Cerebrolysin
P value and statistical significance

| Test | Chi-square | |
|---|---|---|
| Chi-square, df | 0.01290, 1 | |
| z | 0.1136 | |
| P value | 0.9096 | |
| P value summary | ns | |
| One- or two-sided | Two-sided | |
| Statistically significant | No | |
| (P < 0.05)? | | |
| Effect size | Value | 95% CI |
| Relative Risk | 0.8571 | 0.09552 to 7.748 |
| Reciprocal of | 1.167 | 0.1291 to 10.47 |
| relative risk | | |
| Odds ratio | 0.8462 | 0.04167 to 17.41 |
| Reciprocal of odds ratio | 1.182 | 0.05745 to 24.00 |

Methods used to compute CIs

| | | |
|---|---|---|
| Relative Risk | Koopman asymptotic score | |
| Odds ratio | Baptista-Pike | |
| Data analyzed | mRS > 2 | mRS 0-2 |
| Control | 1 | 13 |
| Sovateltide | 1 | 11 |
| Total | 2 | 24 |
| Percentage of row total | mRS > 2 | mRS 0-2 |
| Control | 7.14% | 92.86% |
| Sovateltide | 8.33% | 91.67% |

Referring to FIG. 9 and table 9, a graphical representation and a tabular representation of number of cerebral ischemic stroke patients treated with control and sovateltide along with cerebrolysin and having improvement of 2 or more modified Rankin Scale from baseline at 90 days post-randomization are illustrated, respectively. The number of cerebral ischemic stroke patients with an improvement of 2 or more modified Rankin Scale from baseline at 90 days post-randomization was similar in the group treated with cerebrolysin and an endothelin-B agonist, sovateltide compared to the group treated with cerebrolysin. Therefore, sovateltide did not improve the neurological outcome of cerebrolysin.

TABLE 9
Number of cerebral ischemic stroke patients
treated with control and sovateltide along with
cerebrolysin and having improvement of 2 or more
modified Rankin Scale from baseline at 90 days post-randomization
Change in mRS of 2 or more from day 1 to day 90 Cerebrolysin
P value and statistical significance

| Test | Chi-square | |
|---|---|---|
| Chi-square, df | 0.2243, 1 | |
| z | 0.4736 | |
| P value | 0.6358 | |
| P value summary | ns | |
| One- or two-sided | Two-sided | |
| Statistically significant | No | |
| (P < 0.05)? | | |
| Effect size | Value | 95% CI |
| Relative Risk | 0.9351 | 0.6441 to 1.362 |
| Reciprocal of relative | 1.069 | 0.7343 to 1.553 |
| risk | | |
| Odds ratio | 0.5455 | 0.03485 to 5.316 |
| Reciprocal of odds ratio | 1.833 | 0.1881 to 28.69 |

Methods used to compute CIs

| | | |
|---|---|---|
| Relative Risk | Koopman asymptotic score | |
| Odds ratio | Baptista-Pike | |
| Data analyzed | mRS ≥ 2 | mRS < 2 |
| Control | 12 | 2 |
| Sovateltide | 11 | 1 |
| Total | 23 | 3 |
| Percentage of row total | mRS ≥ 2 | mRS < 2 |
| Control | 85.71% | 14.29% |
| Sovateltide | 91.67% | 8.33% |

Accordingly to another embodiment, a formulation comprising sovateltide and thrombolytics was also prepared. The modified Rankin Scale at post-randomization day 90 of control and sovateltide cerebral ischemic stroke patients (N=29) treated with thrombolytics was determined in 20 patients of the control group and 9 patients in the sovateltide group.

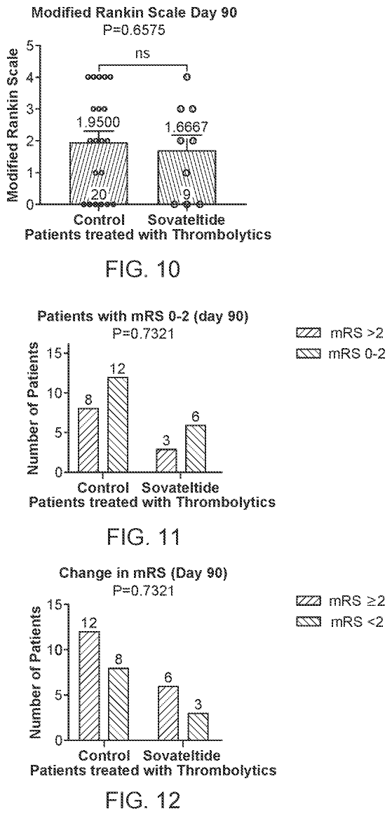

Referring to FIG. 10 and table 10, a graphical and a tabular representation of modified Rankin scale of patients treated with control and sovateltide along with thrombolytics are illustrated, respectively. It was found that patients treated with thrombolytics and an endothelin-B agonist, sovateltide, had similar neurological outcomes compared to those that received thrombolytics.

TABLE 10
Modified Rankin scale of patients treated with
control and sovateltide along with thrombolytics
Modified Rankin Scale Control vs Sovateltide Day 90 Thrombolytics

| | |
|---|---|
| Column B | Sovateltide |
| vs. | vs. |
| Column A | Control |

Unpaired t test

| | |
|---|---|
| P value | 0.6575 |
| P value summary | ns |
| Significantly different (P < 0.05)? | No |
| One- or two-tailed P value? | Two-tailed |
| t, df | t = 0.4483, df = 27 |
| Mean of column A | 1.950 |
| Mean of column B | 1.667 |
| Difference between means (B − A) ± SEM | −0.2833 ± 0.6321 |
| 95% confidence interval | −1.580 to 1.014 |
| R squared (eta squared) | 0.007388 |

Data analyzed

| | |
|---|---|
| Sample size, column A | 20 |
| Sample size, column B | 9 |

Referring to FIG. 11 and table 11, a graphical representation and a tabular representation of number of cerebral ischemic stroke patients treated with control and sovateltide along with thrombolytics and having a modified Rankin Scale of 0-2 at 90 days post-randomization are illustrated, respectively. The number of cerebral ischemic stroke patients with a modified Rankin Scale of 0-2 at 90 days post-randomization was also similar in those treated with thrombolytics and an endothelin-B agonist, sovateltide compared to those with thrombolytic treatment.

TABLE 11
Number of cerebral ischemic stroke patients
treated with control and sovateltide along
with thrombolytics and having a modified Rankin
Scale of 0-2 at 90 days post-randomization
Number of Patients with mRS of 0-2 day 90 Thrombolytics
P value and statistical significance

| Test | Chi-square | |
|---|---|---|
| Chi-square, df | 0.1172, 1 | |
| z | 0.3423 | |
| P value | 0.7321 | |
| P value summary | ns | |
| One- or two-sided | Two-sided | |
| Statistically significant | No | |
| (P < 0.05)? | | |
| Effect size | Value | 95% CI |
| Relative Risk | 1.200 | 0.4685 to 3.664 |
| Reciprocal of relative risk | 0.8333 | 0.2729 to 2.135 |
| Odds ratio | 1.333 | 0.2496 to 5.929 |
| Reciprocal of odds ratio | 0.7500 | 0.1687 to 4.006 |

Methods used to compute CIs

| | | |
|---|---|---|
| Relative Risk | Koopman asymptotic score | |
| Odds ratio | Baptista-Pike | |
| Data analyzed | mRS > 2 | mRS 0-2 |
| Control | 8 | 12 |
| Sovateltide | 3 | 6 |
| Total | 11 | 18 |
| Percentage of row total | mRS > 2 | mRS 0-2 |
| Control | 40.00% | 60.00% |
| Sovateltide | 33.33% | 66.67% |

Referring to FIG. 12 and table 12, a graphical representation and a tabular representation of number of cerebral ischemic stroke patients treated with control and sovateltide along with thrombolytics and having improvement of 2 or more modified Rankin Scale from baseline at 90 days post-randomization are illustrated, respectively. The number of cerebral ischemic stroke patients with an improvement of 2 or more modified Rankin Scale from baseline at 90 days post-randomization was similar in the group treated with thrombolytics and an endothelin-B agonist, sovateltide compared to the group treated with thrombolytics. Therefore, sovateltide did not improve the neurological outcome of thrombolytics.

TABLE 12
Number of cerebral ischemic stroke patients treated
with control and sovateltide along with thrombolytics
and having improvement of 2 or more modified Rankin
Scale from baseline at 90 days post-randomization
Change in mRS of 2 or more from day 1 to day 90 Thrombolytics
P value and statistical significance

| Test | Chi-square | |
|---|---|---|
| Chi-square, df | 0.1172, 1 | |
| z | 0.3423 | |
| P value | 0.7321 | |
| P value summary | ns | |
| One- or two-sided | Two-sided | |
| Statistically significant | No | |
| (P < 0.05)? | | |
| Effect size | Value | 95% CI |
| Relative Risk | 0.9000 | 0.5183 to 1.811 |
| Reciprocal of relative risk | 1.111 | 0.5522 to 1.930 |
| Odds ratio | 0.7500 | 0.1687 to 4.006 |
| Reciprocal of odds ratio | 1.333 | 0.2496 to 5.929 |

Methods used to compute CIs

| | | |
|---|---|---|
| Relative Risk | Koopman asymptotic score | |
| Odds ratio | Baptista-Pike | |
| Data analyzed | mRS ≥ 2 | mRS < 2 |
| Control | 12 | 8 |
| Sovateltide | 6 | 3 |
| Total | 18 | 11 |
| Percentage of row total | mRS ≥ 2 | mRS < 2 |
| Control | 60.00% | 40.00% |
| Sovateltide | 66.67% | 33.33% |

Therefore, the combination of sovateltide and mannitol or sovateltide and citicoline is efficient in treatment of neurological conditions as sovateltide is capable of enhancing the efficacy of mannitol and citicoline resulting in improvement of neurological outcomes and may help treat cerebral stroke, intracranial haemorrhage, brain injury, and other neurological conditions causing cerebral edema.

Although the field has been described herein with limited reference to specific embodiments, this description is not meant to be construed in a limiting sense. Various modifications of the disclosed embodiments, as well as alternate embodiments, will become apparent to persons skilled in the art upon reference to the description.

We claim:

1) A pharmaceutical formulation for treatment of cerebral stroke, comprising:
i) an endothelin-B receptor analog in a dosing range of 0.00001 mg/kg−1 mg/kg; and
ii) a drug in a dosing range of 0.0015 g/kg-5 g/kg.

2) The formulation as claimed in claim 1, wherein said endothelin-B receptor analog includes, but not limited to sovateltide, BQ-3020, [Ala^{1,3,11,15}]-endothelin, sarafotoxin S6c, endothelin-3.

3) The formulation as claimed in claim 1, wherein said drug includes, but not limited to mannitol, citicoline.

4) The formulation as claimed in claim 1, wherein said formulation is a combination of mannitol and sovateltide or citicoline and sovateltide.
